(19) United States
(12) Patent Application Publication
(10) Pub. No.: US 2018/0120318 A1
(43) Pub. Date: May 3, 2018

(54) METHOD OF DIAGNOSIS OF CANCER AND USES THEREOF

(71) Applicant: Goffredo Orazio ARENA, Montreal (CA)

(72) Inventor: Goffredo Orazio ARENA, Montreal (CA)

(21) Appl. No.: 15/565,550

(22) PCT Filed: Sep. 28, 2015
(86) PCT No.: PCT/CA2015/050968

§ 371 Date: Oct. 10, 2017

Related U.S. Application Data
(60) Provisional application No. 62/145,118, filed on Apr. 9, 2015.

(51) Int. Cl. G01N 33/574; G01N 33/50; C12Q 1/6886

(52) U.S. Cl. CPC G01N 33/574; G01N 33/5011; C12Q 1/6886; A61B 10/0041; C12Q 2600/156; C12Q 2600/106; G01N 2800/52

(57) ABSTRACT

The present invention concerns methods of identifying a cancer patient suitable for a treatment, for the treatment of cancer in a patient, for the prognosis of cancer outcome, or of diagnosing a cancer in a patient by determining with a biological assay an oncogenic potential of a sensitized cell contacted with a biological fluid derived from the patient, compared to a reference value, and if the oncogenic potential of the sensitized cell is above or below a reference value, a suitable identification, diagnosis or treatment may be performed.

### CROSS-REFERENCE TO RELATED APPLICATIONS

This application claims priority of U.S. provisional patent application 62/145,118, filed on Apr. 9, 2015, the specification of which is hereby incorporated by reference.

### BACKGROUND

### (a) Field

The subject matter disclosed generally relates to the diagnosis and treatment of cancer. More specifically, the subject matter relates to methods of identifying cancer patients suitable for a treatment and methods of treating cancer in patients.

### (b) Related Prior Art

In Canada, cancer is the leading cause of death being responsible for 30% of all deaths. An estimated 191,000 new cases and 77,000 deaths will happen each year (Canadian Cancer Society, 2015). Despite progress in the understanding of the molecular and genetic basis of this disease, cure or even the 5-year survival rate for some types of cancer has remained very low due to metastatic disease, which is recognized as the prominent cause of cancer-related death. Understanding the mechanisms underlying the metastatic process is the cornerstone to improve cancer patient survival, and such knowledge is needed to develop new prognostic and diagnostic tools. Traditionally, metastasis is described as a multistage process initiated by cancer cells detachment from the primary tumor site, its dissemination via the blood flow, with subsequent homing in distant sites, far from the primary tumor, for the establishment of secondary foci of disease. In this context, research has mainly been focused on the determination of the identity of these Circulating Tumor Cells (CTCs). Nowadays, the detection and molecular characterization of CTCs are one of the most active areas of translational cancer research. If on one hand tremendous increase in the amount of research, examining the potential clinical utility of CTCs in the management of cancer (i.e. detection, diagnosis, prognosis, prediction, stratification, and pharmacodynamics), has been accomplished, on the other hand, the analytical specificity and clinical utility of these detection methods have not been demonstrated unequivocally. Controversies have arisen, since reports from different investigators have shown conflicting results regarding the prognostic relevance of CTCs (Cohen et al, 2009; Rahbari et al, 2010; Tewes et al., 2015; Lalmahomed et al., 2015) and their exploitation as a prognostic marker is still a subject of many ongoing investigations. Furthermore, the lack of correlation between the presence of CTCs and development of metastatic disease has triggered questions regarding the undisputed validity of the “seed and soil” theory.

In the setting of these dubious data, recent and innovative studies have reported that human cancer cells could transfer signaling molecules to target cells predisposing them to malignant transformation (Skoj et al., 2008; Abdel-Mageed et al., 2014; Venugopal et al., 2012). This novel concept, suggests that metastases, might occur via transfer of biologically active circulating factors, (i.e. oncogenes or inhibitors of tumor suppressor genes), derived from the primary tumor, to susceptible target cells located in distant organs, through an activation of survival and mitogenic signals. This alternative theory has been strengthened by the discovery that blood-circulating factors (i.e. cell-free nucleic acids) or factors carried in circulating microvesicles (such as mRNA, micro-RNA, mutated and amplified oncogene sequences and retrotransposon elements) are indeed shed from several types of human tumours and have different biological effects on distinct types of cells. (Grant et al., 2011; Runz et al., 2007; Gaiffe et al., 2012; Hood et al., 2011; Peinado et al., 2012; Balaj et al., 2011; Felischhacker and Schmidt, 2007).

The oncogenic potential of these circulating factors has been first described in immortalized mouse fibroblasts (NIH3T3 cells) and was called “genometastasis” (Garcia-Olmo et al., 1999). More recent studies had brought evidences in favor of this idea and suggested a role of circulating cell-free nucleic acids in the oncogenic transformation of these susceptible murine cells (Garcia-Olmo et al. 2010; Trejo-Becerril et al., 2012). However, in all of these studies, attempts to transform target human cells failed, thus questioning the validity and applicability of this novel and intriguing theory in humans.

The inventor has identified a potential new way of how cancer spreads and metastases occur. It has been discovered that the blood of cancer patients is able to turn healthy cells into cancer cells. This discovery suggests that there are factors circulating in cancer patients that may transmit cancer traits to susceptible cells in other organs leading eventually to their transformation into cancer cells. When normal cells are exposed to the blood of healthy patients no transformation occurs, implying that the factors present in the blood are unique to cancer patients.

Thus, novel methods for diagnosing cancer and/or determining the potential to develop cancer metastasis are highly desirable.

Also, novel methods of predicting the benefit of treatment with are highly desirable.

### SUMMARY

According to an embodiment, there is provided a method of diagnosing a cancer in a patient comprising:
a) determining with a biological assay an oncogenic potential of a sensitized cell contacted with a biological fluid derived from the patient, compared to a reference value; wherein if the oncogenic potential of the sensitized cell is above or below a reference value, the patient may be suitable for the treatment, the patient may be diagnosed as having cancer or a high risk of developing cancer.

According to another embodiment, there is provided a method for treatment of cancer in a patient comprising:
a) determining with a biological assay an oncogenic potential of a sensitized cell contacted with a biological fluid derived from the patient, compared to a reference value; and
b) administering a treatment to the patient if the oncogenic potential of the sensitized cell is above or below a reference value.

According to an embodiment, there is provided a method for the prognosis of cancer outcome, comprising:
a) determining with a biological assay an oncogenic potential of a sensitized cell contacted with a biological fluid derived from the patient, compared to a reference value;
   wherein when the oncogenic potential is above the predetermined reference value, prognosis of the cancer outcome may be a bad prognosis; and
   wherein when the oncogenic potential is below the predetermined reference value, prognosis of the cancer outcome may be a good prognosis.

According to an embodiment, there is provided a method of identifying a cancer patient suitable for a treatment comprising:
a) determining with a biological assay an oncogenic potential of a sensitized cell contacted with a biological fluid derived from the patient, compared to a predetermined reference value; wherein if the oncogenic potential of the sensitized cell is above or below a reference value, the patient may be suitable for the treatment.

The sensitized cell may be chosen from an immortalized cell, a normal cell with a single oncosuppressor gene mutation, a normal cell with a single oncosuppressor gene decreased gene expression, a normal cell with a single activating mutation in a protooncogene, a normal cell with a single protooncogene increased gene expression.

The immortalized cell may be a HEK293 cell.

The normal cell with a single oncosuppressor gene mutation may be a BRCA mutated fibroblast.

The normal cell with a single oncosuppressor gene decreased gene expression may be a fibroblast with a decrease BRCA expression.

The oncogenic potential of the sensitized cell may be above the reference value

The oncogenic potential of the sensitized cell may be below the reference value

The biological fluid derived from the patient may be chosen from blood, serum, lymph, and a culture media contacted with a tumor from the patient.

The biological assay may be a soft agar colony formation/anchorage independent cell growth assay, an in vivo tumor growth assay, a cellular growth rate measurement assay, a cellular metabolic rate measurement assay, a cellular proliferation rate measurement assay, a biomarker expression measurement assay, a biomarker activity measurement assay, an exosome internalization assay, or a combination thereof.

The soft agar colony formation/anchorage independent cell growth assay provides an increase of colony size of the sensitized cells contacted with the biological fluid derived from the patient, compared to a reference value from a control.

The soft agar colony formation/anchorage independent cell growth assay provides an increase of the number of colonies of the sensitized cells contacted with the biological fluid derived from the patient, compared to a reference value from a control.

The in vivo tumor growth assay provides an increased tumor diameter, an increased tumor volume, or both, at a given time, of the sensitized cells contacted with the biological fluid derived from the patient, compared to a reference value from a control at the given time.

The cellular growth rate measurement assay provides an increased growth rate of the sensitized cell contacted with the biological fluid derived from the patient, compared to a reference value from a control.

The cellular metabolic rate measurement assay provides an increased metabolic activity of the sensitized cell contacted with the biological fluid derived from the patient, compared to a reference value from a control.

The cellular proliferation rate measurement assay provides an increased proliferation of the sensitized cell contacted with the biological fluid derived from the patient, compared to a reference value from a control.

The biomarker expression measurement assay provides an increased expression of a biomarker or a decreased expression of a biomarker in the sensitized cell contacted with the biological fluid derived from the patient, compared to a reference value from a control.

The biomarker activity measurement assay provides an increased activity of a biomarker or a decreased activity of a biomarker in the sensitized cell contacted with the biological fluid derived from the patient, compared to a reference value from a control.

The treatment may be chosen from a surgical intervention, administering a therapeutic agent, a radiotherapy treatment, and a combination thereof.

The cancer may be selected from the group consisting of breast cancer, colon cancer, pancreatic cancer, sarcoma, prostate cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, kidney cancer, bladder cancer, neuroendocrine tumor and liver cancer.

The method of the present invention may further comprise a step of cancer screening.

The cancer screening may be a serum tumor marker screening, a colonoscopy, a mammogram, a prostate exams, a PET scan, a CT scan, an MRI scan, an ultrasound scan, or combinations thereof.

The patient may be a patient having had a primary tumor resected.

According to another embodiment, there is provided a kit for performing the method of the present invention, comprising:
a) a sensitized cell,
b) instructions on how to perform the method.

The following terms are defined below.

The term “sensitized cell” or “sensitized cell line” is intended to mean a cell or cell line that has the genetic or molecular characteristics (i.e. modifications, mutations, or any other premalignant lesions) that could lead to eventual malignant transformation of the cells and become oncogenic. Examples of such cells or cell lines include but are not limited to immortalized cells, normal cell with a single oncosuppressor gene mutation, normal cell with a single oncosuppressor gene decreased gene expression, normal cells with a single activating mutation in a proto-oncogene, or normal cells with a single proto-oncogene increased gene expression. Preferably, the cell or cell line may be a human embryonic kidney cell line (HEK293), which is immortalized following culture with shared Adenovirus 5 DNA (Louis et al., 1997) Also preferred are human fibroblast with a single oncosuppressor mutation or human mesenchymal stem cells with a single oncosuppressor mutation.

The term “biological fluid” or “biological fluid derived from the patient” is intended to mean any suitable biological fluid which may be obtained from the patient directly, for example through a blood draw, or similar collections. Alternatively, it may also be fluid derived from a tissue sample from the patient, for example through incubation of a tissue sample of the patient in a culture media. Suitable fluids include blood, serum, and lymph. Examples also include culture media contacted with a tumor from the patient.

The term “biological assay” is intended to mean any suitable biological assay that could indicate that the cell or cell line tested acquires oncogenic potential (as defined below).

The term “oncogenic potential” is intended to mean that the cell or cell line tested may or may not have the potential to cause cancer, compared, for example, to a certain reference state. Depending on the assay used to assess such potential, the result of the assay may be a binary value, such as a “yes” or a “no”, indicative that the cell will cause cancer, or will not cause, or will or will not turn into cancer, which may be then used as indications that the patient may be considered as having a low chance of having cancer, or even to be cancer or tumor free, or that the patient may be considered as having a high chance or risk of having cancer or developing cancer, or that the patient may be considered as having a high chance of having cancer anew and/or cancer metastasis. According to other embodiment, the oncogenic potential calculated may be a number or value, which may be higher or lower than a control value, which will be indicative that the cell may or will cause cancer, or may or will not cause cancer, or may or will or may or will not turn into cancer, which may be then used as indications that the patient may be considered as having a low chance of having cancer, or even to be cancer or tumor free, or that the patient may be considered as having a high chance or risk of having cancer or developing cancer, or that the patient may be considered as having a high chance of having cancer anew and/or cancer metastasis. According to yet another embodiment, the oncogenic potential calculated may be a number or value, which may be higher or lower than a control value, which will be indicative that the cell has a certain chance or odd of causing cancer, or of not causing cancer, or will or will not turn into cancer, which may be then used as indications that the patient may be considered as having a low chance of having cancer, or even to be cancer or tumor free, or that the patient may be considered as having a high chance or risk of having cancer or developing cancer, or that the patient may be considered as having a high chance of having cancer anew and/or cancer metastasis.

The terms “reference value” or “reference condition” is intended to mean a value or condition relative to which the oncogenic potential of the sensitized is determined, and which represent a normal state, a basal state, an untreated state, a sensitized cell having been treated with a biological fluid that does not cause the so called transformation of the sensitized cells (i.e. a negative control). The reference value or condition is in essence a state to which a positive oncogenic potential is assessed. In some embodiments, the reference value or condition is obtained from a biological assay at the same time that the biological fluid from a patient is tested for its oncogenic potential. According to another embodiment, the reference value or condition is predetermined, for example having been obtained from previous experiments. According to another embodiment, the reference value or condition may be obtained from calculations from all experimental results, for example from averaged normalized values obtained from a given biological assays.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates that cancer patient serum increased 293 cells growth. 293 cells were cultured for 3 weeks in control human serum, or cancer patient sera (A-C). Cells were than analyzed for their growth potential. (A) population doublings capability was calculated at every passage. Column graphs represent cumulative population doublings at the end of the treatment periods. (B) metabolic activity following 6 hours incubation with Alamar Blue and spectrofluorometry analyses. (C) proliferation following labeling with CFSE probe and cytometry acquisition. Numbers in brackets are the mean fluorescence intensity (MFI) of each peak. Data are mean±SD of 2 control sera vs. 4 cancer patient sera (A-C).

FIG. 2 illustrates that cancer patient serum increased anchorage-independent growth of 293 cells. 293 cells were cultured for 3 weeks in control human serum, or cancer patient sera (A-C). Cells were then grown in soft agar for 2 weeks. (A; Bright field pictures), note the increase of colonies size in the cells exposed to patient sera compared to control. (B) The graphs represent the number of colonies counted per field. (C; Colony size distribution) the sizes of the colonies were measured using ImageJ™ software and the frequency of different colony size was calculated. Note that the biggest colonies are formed in the cells exposed to cancer patient sera. Data are mean±SD of 2 control sera vs. 4 cancer patient sera.

FIG. 3 illustrates the effect of cancer patient serum on tumorigenicity of 293 cells in vivo. SCID/Beige mice were injected with 293 cells cultured for 3 weeks in control human serum, or cancer patient sera. (A) 4 to 5 weeks after injection, mice were photographed and euthanized. Representative pictures of tumors are shown. (B and C) tumor growth was monitored weekly. Once tumors were palpable, their diameters were measured (B) and their volumes at euthanasia were calculated (C). Values are mean+/−SD, (n=3-6 mice per group).

FIG. 4 illustrates the in vivo growth of human fibroblasts infected with empty plasmid (PX458) or plasmid carrying a single guided BRCA1 (sg BRCA1) construct BRCA-1 to knock down the oncosuppressor gene BRCA-1. PHS stands for pooled human serum and Case219 stands for serum from patient 219.

FIG. 5 illustrates the effect of cancer patient serum on tumorigenicity of BRCA-1 knock down human fibroblasts in vivo. SCID/Beige mice were injected with BRCA-1 knock down human fibroblasts cultured for 3 weeks in control human serum, or cancer patient sera. (A) 4 to 5 weeks after injection, mice were photographed and euthanized. Representative pictures of tumors are shown. (B) tumor growth was monitored weekly. Once tumors were palpable, their volumes were calculated at euthanasia. Values are mean +/−SD, (n=3-6 mice per group).

FIG. 6 illustrates the effect of cancer patient serum on tumorigenicity of 293 cells in vivo. SCID/Beige mice were injected with 293 cells cultured for 3 weeks in control human serum, or cancer patient sera. (A) 4 to 5 weeks after injection, mice were photographed and euthanized. Representative pictures of tumors are shown. (B) tumor growth was monitored weekly. Once tumors were palpable, their volumes were calculated at euthanasia. Values are mean+/−SD, (n=3-6 mice per group).

FIG. 7 illustrates the internalization of exosomes by sensitized cells. (A) Staining with PKH-26 (red) shows the stained exosomes in HEK293 cells, fibroblasts infected with sgBRCA1 or empty PX458 vector, Also shown is nuclear DNA stained with DAPI; (B) Quantification of the color intensity using the ImageJ software for PX458-fibroblasts, sgBRCA1 fibroblasts, and HEK293 cells treated or not with patient serum; (C) Quantification of the area using the ImageJ software for PX458-fibroblasts, sgBRCA1 fibroblasts, and HEK293 cells treated or not with patient serum. The results show that the sensitized cells exposed to cancer serum internalized a greater number of cancer exosomes, suggesting, at least for the sgBRCA1 that oncosuppressor genes act by protecting cells from internalizing outside material that can induce genome instability.

FIG. 8 illustrates a putative pathway explaining metastatic disease and where the present invention acts to discover cancer presence in the body and metastatic risk.

### DETAILED DESCRIPTION

Primary cells (i.e. “normal” primary cells) such as human embryonic stem cells (hESCs), human mesenchymal stem cells (hMSCs) and human adult liver fibroblasts (hALFs) have been exposed to serum of patients with metastatic cancer in order to “transform” them, but repeated attempts were consistently unsuccessful (Garcia-Olmo et al. 2010; Trejo-Becerril et al., 2012; Abdouh et al 2014). To explain this discrepancy between results in humans and mice, it was hypothesized that human target cells must be firstly “primed” or “sensitized” prior to exposure to cancer patient serum to be able to transform. The premise for this hypothesis finds its rationale in the proven concept that in the clinical settings, malignant transformation of normal human cells is a multistep process where genetic changes are accumulated, thus progressively transforming cells into a cancerous phenotype. The underlying molecular mechanisms involve the co-expression of cooperating oncogenes, “two hit hypothesis”, which eventually lead to the malignant transformation of a normal cell after transiting through the stage of premalignant lesion.

To test this assumption, human embryonic kidney cell line (HEK293), which is immortalized following culture with shared Adenovirus 5 DNA (Louis et al., 1997) was used. This cell line is not oncogenic but it was shown to be prone to malignant transformation following in vitro transfer of oncogenes (Ha et al. 2010; Hamid et al., 2005; Lin et al., 2011; Canis et al., 2013), and can thus be regarded as a “primed” or “sensitized cell” line. Due to these characteristics, the HEK293 cells represent a good model of a human cell, which albeit not oncogenic, has the potential to become cancerous if exposed to a presumed oncogenic stimulation carried through the blood.

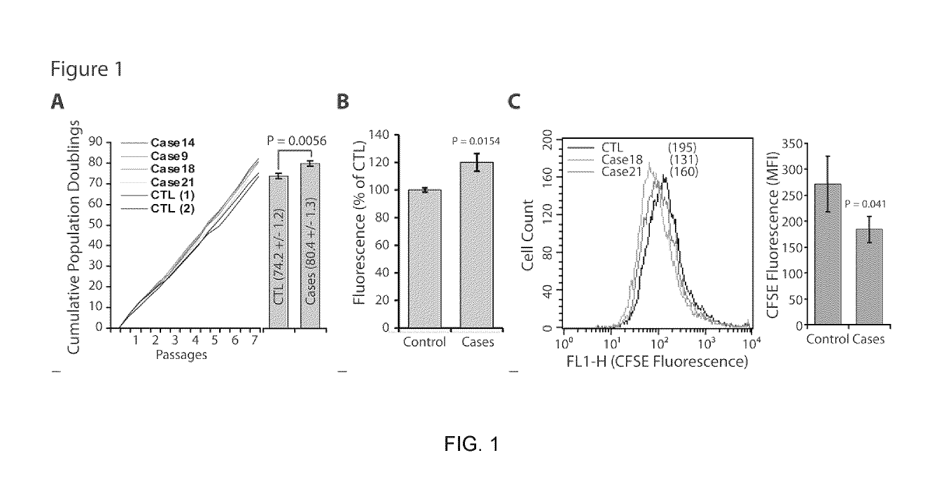

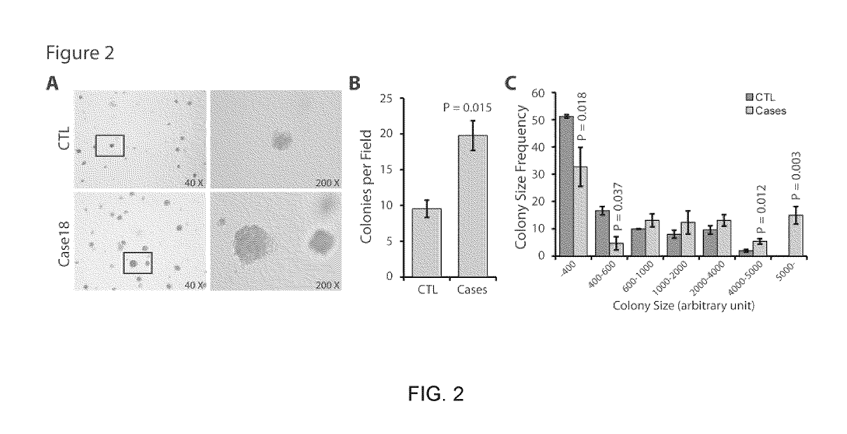

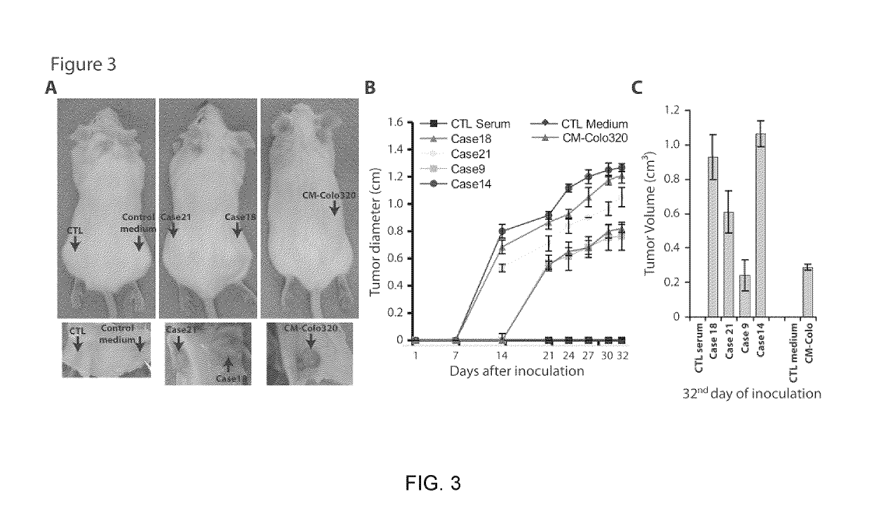

Treated HEK293 cells displayed characteristics of transformed cells following exposure to metastatic cancer patient sera (Abdouh et al., 2014). Independently of the type of cancer, these experiments confirmed that metastatic cancer patient sera significantly enhanced the proliferation of HEK293 cells in vitro. Cell proliferation was quantified by analyzing population doubling potential (FIG. 1A), cell metabolic activity (Alamar blue assay; FIG. 1B) and cell division (CFSE label dilution; FIG. 1C). Furthermore HEK293 treated with cancer patient sera were used to perform anchorage-independent growth assay, which is a hallmark for cancer cells. With all cancer patient sera that were tested (breast cancer, colon cancer, pancreatic cancer and sarcoma), HEK293 cells gave rise to more and larger colonies than compared to those generated by cells grown in control human serum (FIG. 2). These results suggest that cancer patient sera may contain oncogenic factors, which have the ability to transform HEK293 cells in vitro. To determine whether cancer patient sera promote tumor formation in vivo, NOD/SCID mice were injected subcutaneously with HEK293 cells exposed to control or cancer patient sera (FIG. 3A). All mice injected with cancer sera-treated cells developed visible tumors as early as 2 weeks following inoculation (FIG. 3B). These tumors vary in size from 0.24 to 1.06 cm³ (FIG. 3C). The same phenotypes were acquired when these cells were cultured in cancer cell line conditioned medium, suggesting that the putative oncogenic factors present in the human serum might derive directly from the primary tumor. In contrast, none of the mice injected with control human serum-treated cells developed tumors during the course of the experiments (5 weeks latency) (FIGS. 3A-C).

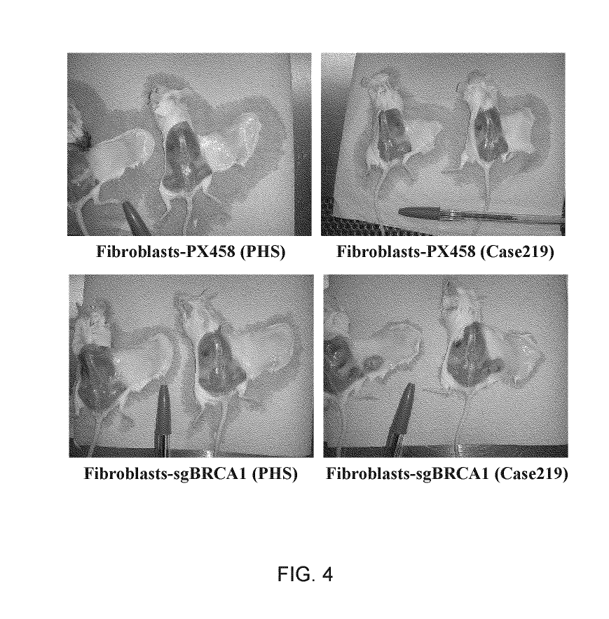

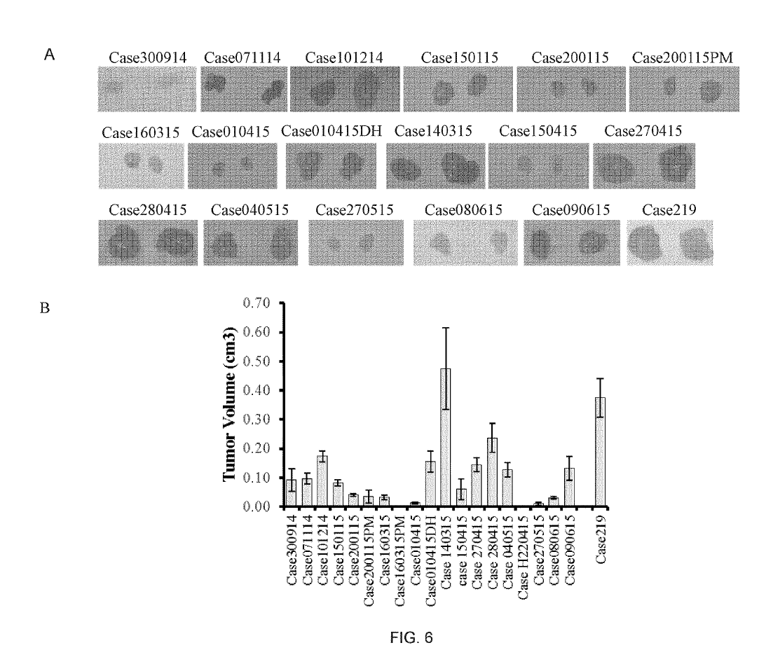

These experiments were repeated using again HEK293 cells as well as fibroblasts where BRCA-1 is knocked down using a single guided (sg) RNA. The results obtained with the knock down fibroblasts was the same results given by HEK293 (See FIGS. 4-6). These results suggest that any human cell with a single oncosupressor mutation can be used for a screening test according to the present invention.

Altogether, those data suggest that human cancer sera transfer tumorigenic traits in vitro and in vivo to an immortalized human cell line or a normal cell with a single oncosuppressor gene decreased gene expression, and confirm for the first time the validity of the genometastatic theory in human cells. When this novel HEK293/fibroblast BRCA mutated based platform was tested with sera of 2 patients drawn prior to surgical resection, the response was quite enticing since the HEK293 cells turned malignant, even when they were exposed to sera of these 2 patients with normal tumor markers and whose pathological stage was found to be T₁, N₀ and T₂, N₀.

In embodiments there is disclosed a method of identifying a cancer patient suitable for a treatment comprising:
a) contacting a sensitized cell with a biological fluid derived from the patient;
b) determining with a biological assay an oncogenic potential of the sensitized cell, compared to a reference value; and
c) identifying the patient as suitable for the treatment if the oncogenic potential of the sensitized cell is above or below the reference value.

In another embodiment, there is disclosed a method of identifying a cancer patient suitable for a treatment comprising:
a) determining with a biological assay an oncogenic potential of a sensitized cell contacted with a biological fluid derived from the patient, compared to a reference value; and
b) identifying the patient as suitable for the treatment if the oncogenic potential of the sensitized cell is above or below a reference value.

In another embodiment, there is disclosed a method of identifying a cancer patient suitable for a treatment comprising:
a) determining with a biological assay an oncogenic potential of a sensitized cell contacted with a biological fluid derived from the patient, compared to a reference value; wherein if the oncogenic potential of the sensitized cell is above or below a reference value, the patient is suitable for the treatment.

In another embodiment, there is disclosed a method for treatment of cancer in a patient comprising:
a) contacting a sensitized cell with a biological fluid derived from the patient;
b) determining with a biological assay an oncogenic potential of the sensitized cell, compared to a reference value; and
c) administering a treatment to the patient if the oncogenic potential of the sensitized cell is above or below a reference value.

In another embodiment, there is disclosed a method for treatment of cancer in a patient comprising:
a) determining with a biological assay an oncogenic potential of a sensitized cell contacted with the biological fluid derived from the patient, compared to a reference value; and
b) administering a treatment to the patient if the oncogenic potential of the sensitized cell is above or below a reference value.

In another embodiment, there is disclosed a method for the prognosis of cancer outcome, comprising:
a) contacting with a biological fluid derived from the patient;
b) determining with a biological assay an oncogenic potential of the sensitized cell, compared to a reference value;

Under these circumstances, when the oncogenic potential is above the reference value, prognosis of the cancer outcome is a bad prognosis; and when the oncogenic potential is below the reference value, prognosis of the cancer outcome is a good prognosis.

In another embodiment, there is disclosed a method for the prognosis of cancer outcome, comprising:
a) determining with a biological assay an oncogenic potential of a sensitized cell contacted with the biological fluid derived from the patient, compared to a reference value;
   and
b) identifying the patient as suitable for the treatment if the oncogenic potential of the sensitized cell is above or below a reference value.

Under these circumstances, when the oncogenic potential is above the reference value, prognosis of the cancer outcome is a bad prognosis; and when the oncogenic potential is below the reference value, prognosis of the cancer outcome is a good prognosis.

In another embodiment, there is disclosed a method of diagnosing a cancer in a patient comprising:
a) contacting a sensitized cell with a biological fluid derived from the patient;
b) determining with a biological assay an oncogenic potential of the sensitized cell, compared to a reference value; and
c) diagnosing the patient as having cancer or high risk to develop cancer if the oncogenic potential of the sensitized cell is above or below the reference value.

In another embodiment, there is disclosed a method of diagnosing a cancer in a patient comprising:
a) determining with a biological assay an oncogenic potential of a sensitized cell contacted with a biological fluid derived from the patient, compared to a predetermined reference value; and
b) diagnosing the patient as having cancer or a high risk to develop cancer if the oncogenic potential of the sensitized cell is above/below the reference value.

In another embodiment, there is disclosed a method of diagnosing a cancer in a patient comprising:
a) determining with a biological assay an oncogenic potential of a sensitized cell contacted with a biological fluid derived from the patient, compared to a reference value; wherein if the oncogenic potential of the sensitized cell is above or below a reference value, the patient is suitable for the treatment, the patient is diagnosed as having cancer or a high risk of developing cancer.

According to an embodiment, as used herein, the sensitized cell or cell line may be chosen from an immortalized cell, a normal cell with a single oncosuppressor gene mutation, a normal cell with a single oncosuppressor gene decreased gene expression, a normal cell with a single activating mutation in a protooncogene, a normal cell with a single protooncogene increased gene expression. Preferably, the cell or cell line is a human embryonic kidney cell line (HEK293) or BRCA mutated/knocked down fibroblast. For example, the normal cell may have been engineered through replacement of the normal alleles of an oncosuppressor gene or a protooncogene with a mutated one, or the normal cell may have been engineered through knock-out (through homologous recombination or genome editing [i.e. with CRISPR]), or knock-down using well known technologies such as siRNA, shRNA, antisense RNA/DNA, and the likes, or engineered through increased (often termed overexpression) of the protooncogene through means well known in the art.

According to an embodiment biological fluid derived from the patient may be chosen from blood, serum, lymph, and a culture media contacted with a tumor from the patient. These biological fluids may be collected using routine techniques well known to the person skilled in the art. The biological fluid may be added to the culture medium of the sensitized cell according to known cell culture practices, and replenished over time as may be needed to obtain the cells necessary to confirm or infirm the transformed phenotype.

According to an embodiment, the cancer may be breast cancer, colon cancer, pancreatic cancer and sarcoma. According to another embodiment, the cancer may be prostate cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, kidney cancer, bladder cancer, neuroendocrine tumor and liver cancer.

According to an embodiment the biological assay used in the methods of the present invention may be any suitable assay. Examples of assays that have been successfully used for the present invention include soft agar colony formation/anchorage independent cell growth assay, an in vivo tumor growth assay, in which the tested cells were shown to develop as tumors compared to cells that had not been treated with biological fluids from patients having cancer, a cellular growth rate measurement assay, a cellular metabolic rate measurement assay, a cellular proliferation rate measurement assay, a biomarker expression measurement assay, a biomarker activity measurement assay.

According to an embodiment, anchorage-independent cell growth may be determined by analyzing the formation of colonies in soft agar. This in vitro assay is a hallmark of transformed cells. It determines the (i) incidence of colony formation, that is the frequency of cells able to grow and form colonies, and (ii) size distribution of these colonies, that reflects growth speed of cells in a given colony (i.e. the faster the cells grow, the bigger the colony they form). For this purpose, the size of all colonies in a given culture condition may be determined using an imaging technique, such as analysis with ImageJ™ Software. The values obtained are then categorized to compare one culture condition to another (i.e. treatment with serum from a cancerous patient vs. a normal patient). See FIG. 2 for example. For example, the soft agar colony formation/anchorage independent cell growth assay may provide an increase of colony size of the sensitized cells contacted with the biological fluid derived from the patient, compared to a reference value from a control. The soft agar colony formation/anchorage independent cell growth assay may provide an increase of the number of colonies of the sensitized cells contacted with the biological fluid derived from the patient, compared to a reference value from a control.

According to another embodiment, in vivo tumor growth may be tested in NOD-SCID mice. These animals are homozygous for the SCID mutation and have impaired T and B cell lymphocyte development. The NOD background additionally results in deficient natural killer (NK) cell function. Sensitized or control cells growing in log phase are harvested by trypsinization and washed twice with HBSS and injected subcutaneously in the mice. Tumor growth is then monitored regularly in all animals and once palpable masses were detected, the diameter was recorded with a caliper and volume estimated using the following formula V=a×b²×(π/6) (where a=major diameter; b=minor diameter and V=volume). See FIG. 3 for example. According to an embodiment, the in vivo tumor growth assay may provide an increased tumor diameter, an increased tumor volume, or both, at a given time, of the sensitized cells contacted with the biological fluid derived from the patient, compared to a reference value from a control at the same given time.

According to an embodiment, the cellular growth rate measurement assay may provide an increased growth rate of the sensitized cell contacted with the biological fluid derived from the patient compared to a reference value from a control. Also, the cellular metabolic rate measurement assay may provide an increased metabolic activity of the sensitized cell contacted with the biological fluid derived from the patient, compared to a reference value from a control. Also, the cellular proliferation rate measurement assay may provide an increased proliferation of the sensitized cell contacted with the biological fluid derived from the patient, compared to a reference value from a control.

According to another embodiment, the biological assay may be the measurement of the expression and or presence of a known or novel biomarker associated with cancer, in the sensitized cells. According to one embodiment, the biomarker expression may be measured with a biomarker expression measurement assay such as quantitative PCR, DNA or protein expression arrays, quantitative western blotting, or the likes. According to embodiments, the biomarker expression measurement assay may provide increased expression of the biomarker or a decreased expression of the biomarker in the sensitized cell contacted with the biological fluid derived from the patient, compared to a reference value from a control, such for example sensitized cells treated with a sera from a normal patient.

According to another embodiment, the biological assay may be the measurement of the activity of a known or novel biomarker associated with cancer, in the sensitized cells. According to one embodiment, the biomarker activity may be measured with a biomarker activity measurement assay such as metabolite processing assays of the biomarkers as a measurable enzymatic metabolite processing activity, kinase assay, if the biomarker as such a kinase activity, phosphorylation status, if the biomarker may be activated or deactivated through phosphorylation, detection or the presence or the absence of an antibody, or the likes. According to embodiments, the biomarker expression measurement assay may provide increased expression of the biomarker or a decreased expression of the biomarker in the sensitized cell contacted with the biological fluid derived from the patient, compared to a reference value from a control, such for example sensitized cells treated with a sera from a normal patient.

According to yet another embodiment, the biological assay may be the assessment of an increase in the internalization of exosomes, particularly cancer exosomes into the sensitized cell. For example, the internalization of exosomes may results in more intense staining with markers such as PHK26-red, which can be assessed through measurement of fluorescence intensity in the sensitized cells, as well as through measurement of the stained area in the sensitized cells.

The biological assays described above allow the determination of the oncogenic potential of the sensitized cell, compared to a reference condition, such as control cells treated with serum from a healthy individual. The determination involves a number of measurements and calculations such as growth rates, metabolic rates, proliferation rates, colony sizes, colony numbers, tumor volume and/or diameters, biomarker expression (increase or decrease), biomarker activity (increase or decreases), exosome internalization (increase or decrease) and the likes. The calculated value will allow the skilled person to determine if the sensitized cells treated with the biological fluid have a positive oncogenic potential (i.e. associated with causing cancer) or a negative one (i.e. associated with not causing cancer). For example, according to some embodiments, the person skilled in the art would understand that an increase in growth rates, metabolic rates, proliferation rates, colony sizes, colony numbers, tumor volume and/or diameters, increased exosome internalization for the sensitized cell contacted with the biological fluid derived from the patient, compared to a reference value from a control provides a positive oncogenic potential (i.e. associated with causing cancer), while any such values decreased or equal to the reference values represent negative oncogenic potential (i.e. associated with not causing cancer).

The determination may also involve a number of measurements and calculations such as biomarker expression (increase or decrease), biomarker activity (increase or decreases). The person skilled in the art will appreciate that the correlation between the biomarker's expression and/or activity and cancer will vary according to the role of the biomarkers. For example, growth promoters increases or decreases may be expected to correlate with increases or decreases in oncogenic potential respectively. Likewise, growth suppressors increases or decreases may be expected to correlate with decreases or increases in oncogenic potential respectively.

According to embodiment, the method of the present invention may be used to determine the suitability of a patient to a given treatment. This method may be used at the primary and tertiary prevention level.

At the primary level, the biological fluid of a patient may be collected and tested according to the steps described above to assess if the patient has cancer or has an increased risk of developing a cancer. According to an embodiment, if the method is performed and the oncogenic potential is determined to be low (or negative) (e.g. the cells do not display increase growth rates and colony sizes, and do not form, or form only small tumors compared to a positive control in vivo) these results are used as indications that the patient may be considered as having a low chance of having cancer, or even to be cancer or tumor free. According to another embodiment, if the method is performed and the oncogenic potential is determined to be high (or positive) (e.g. the cells do display increase growth rates and colony sizes, and do form tumors compared to a normal control in vivo) these results are used as indications that the patient may be considered as having a high chance or risk of having cancer or developing cancer. Based on such oncogenic potential, the patient may be determined to be suitable for a treatment for his cancer. As used herein, the term treatment is intended to involve, as may be necessary, any suitable screening tests known in the art, such as such as serum tumor markers, colonoscopy, mammograms, prostate exams, PET, CT scans, MRI scans such as full body MRI, ultrasound scans and the likes, to identify the exact nature of the cancer. Depending on the diagnosis obtained from these tests, further treatment may be adequately prescribed to the patient.

At the tertiary level, the biological fluid of a patient having had a primary tumor resected may be subjected to the method of the present invention to assess if the patient is likely develop cancer anew, for example cancer metastasis, or if the patient has already developed a new cancer or cancer metastasis. According to an embodiment, if the method is performed and the oncogenic potential is determined to be low (or negative) (e.g. the cells do not display increase growth rates and colony sizes, and do not form, or form only small tumors compared to a positive control in vivo) these results are used as indications that the patient may be considered as having a low chance of having cancer, or even to be cancer or tumor free. Under such circumstances, the physician may determine that the patient would not need to be subjected to some preventive treatment that would normally be administered, if the information was not otherwise available. For example, this may avoid the patient being subjected to an unnecessary chemotherapeutic treatment.

According to another embodiment, if the method is performed and the oncogenic potential is determined to be high (or positive) (e.g. the cells do display increase growth rates and colony sizes, and do form tumors compared to a normal control in vivo) these results are used as indications that the patient may be considered as having a high chance of having cancer anew and/or cancer metastasis. Based on such oncogenic potential, the patient may be determined to be suitable for a treatment for his cancer. As used herein, the term treatment is intended to involve, as may be necessary, any suitable screening tests known in the art, such as serum tumor markers, colonoscopy, mammograms, prostate exams, PET, CT scans, MRI scans such as full body MRI, ultrasound scans and the likes, to identify the exact nature of the cancer. Depending on the diagnosis obtained from these tests, further treatment may be adequately prescribed to the patient. Immediately or after further testing, the patient may be subjected to a cancer treatment.

According to an embodiment, the treatment may be chosen from a surgical intervention, administering a therapeutic agent, and a combination thereof.

The methods of the invention may also be used in combination with radiotherapy in the treatment of cancer.

The therapeutic agent may be one or more anticancer agents selected from cytotoxic agents, mitotic poisons, anti-metabolites, proteasome inhibitors and kinase inhibitors, and to the use of that type of combination in the manufacture of medicaments for use in the treatment of cancer.

Therapeutic agents also include, but are not limited to, angiogenesis inhibitors, antiproliferative agents, other kinase inhibitors, other receptor tyrosine kinase inhibitors, aurora kinase inhibitors, polo-like kinase inhibitors, bcr-abl kinase inhibitors, growth factor inhibitors, COX-2 inhibitors, EP4 antagonists, non-steroidal anti-inflammatory drugs (NSAIDS), antimitotic agents, alkylating agents, antimetabolites, intercalating antibiotics, platinum containing agents, growth factor inhibitors, ionizing radiation, cell cycle inhibitors, enzymes, topoisomerase inhibitors, biologic response modifiers, immunologicals, antibodies, hormonal therapies, retinoids/deltoids plant alkaloids, proteasome inhibitors, HSP-90 inhibitors, histone deacetylase inhibitors (HDAC) inhibitors, purine analogs, pyrimidine analogs, MEK inhibitors, CDK inhibitors, ErbB2 receptor inhibitors, mTOR inhibitors, Bcl inhibitors, Mcl inhibitors and combinations thereof as well as other antitumor agents.

Angiogenesis inhibitors include, but are not limited to, EGFR inhibitors, PDGFR inhibitors, VEGFR inhibitors, TTE2 inhibitors, IGFIR inhibitors, matrix metalloproteinase 2 (MMP-2) inhibitors, matrix metalloproteinase 9 (MMP-9) inhibitors, thrombospondin analogs such as thrombospondin-1 and N-Ac-Sar-Gly-Val-D-allolle-Thr-Nva-Ile-Arg-Pro-NHCH₂CH₃ or a salt thereof and analogues of N-Ac-Sar-Gly-Val-D-allolle-Thr-Nva-Ile-Arg-PrO-NHCH₂CH₃ such as N-Ac-GlyVal-D-alle-Ser-Gln-Ile-Arg-ProNHCH₂CH₃ or a salt thereof.

Examples of EGFR inhibitors include, but are not limited to, Iressa (gefitinib), Tarceva (erlotinib or OSI-774), Icotinib, Erbitux (cetuximab), EMD-7200, ABX-EGF, HR3, IgA antibodies, TP-38 (IVAX), EGFR fusion protein, EGF-vaccine, anti-EGFr immunoliposomes and Tykerb (lapatinib).

Examples of PDGFR inhibitors include, but are not limited to, CP-673,451 and CP-868596.

Examples of VEGFR inhibitors include, but are not limited to, Avastin (bevacizumab), Sutent (sunitinib, SUI 1248), Nexavar (sorafenib, BAY43-9006), CP-547,632, axitinib (AG13736), Apatinib, cabozantinib, Zactima (vandetanib, ZD-6474), AEE788, AZD-2171, VEGF trap, Vatalanib (PTK-787, ZK-222584), Macugen, M862, Pazopanib (GW786034), ABT-869, BC-00016 and angiozyme.

Examples of thrombospondin analogs include, but are not limited to, ABT-510.

Examples of BCL inhibitors include, but not limited to, ABT263, ABT199 and GX-015.

Examples of aurora kinase inhibitors include, but are not limited to, VX-680, AZD-1152 and MLN-8054. Example of polo-like kinase inhibitors include, but are not limited to, BI-2536.

Examples of bcr-abl kinase inhibitors include, but are not limited to, Gleevec (imatinib) and Dasatinib (BMS354825).

Examples of platinum containing agents includes, but are not limited to, cisplatin, Paraplatin (carboplatin), eptaplatin, lobaplatin, nedaplatin, Eloxatin (oxaliplatin) or satraplatin.

Examples of mTOR inhibitors includes, but are not limited to, CCI-779, rapamycin, temsirolimus, everolimus, RAD001, INK-128 and ridaforolimus.

Examples of HSP-90 inhibitors includes, but are not limited to, geldanamycin, radicicol, 17-AAG, KOS-953, 17-DMAG, CNF-101, CNF-1010, 17-AAG-nab, NCS-683664, Mycograb, CNF-2024, PU3, PU24FC1, VER49009, IPI-504, SNX-2112 and STA-9090.

Examples of histone deacetylase inhibitors (HDAC) includes, but are not limited to, Suberoylanilide hydroxamic acid (SAHA), MS-275, valproic acid, TSA, LAQ-824, Trapoxin, tubacin, tubastatin, ACY-1215 and Depsipeptide.

Examples of MEK inhibitors include, but are not limited to, PD325901, ARRY-142886, ARRY-438162 and PD98059.

Examples of CDK inhibitors include, but are not limited to, flavopyridol, MCS-5A, CVT-2584, seliciclib (CYC-202, R-roscovitine), ZK-304709, PHA-690509, BMI-1040, GPC-286199, BMS-387,032, PD0332991 and AZD-5438.

Examples of COX-2 inhibitors include, but are not limited to, celecoxib, parecoxib, deracoxib, ABT-963, etoricoxib, lumiracoxib, BMS347070, RS 57067, NS-398, valdecoxib, paracoxib, rofecoxib, SD-8381, 4-Methyl-2-(3,4-dimethylphenyl)-1-(4-sulfamoyl-phenyl-1H-pyrrole, T-614, JTE-522, S-2474, SVT-2016, CT-3, SC-58125 and etoricoxib.

Examples of non-steroidal anti-inflammatory drugs (NSAIDs) include, but are not limited to, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol) and Oxaprozin (Daypro).

Examples of ErbB2 receptor inhibitors include, but are not limited to, CP-724-714, CI-1033, (canertinib), Herceptin (trastuzumab), Omitarg (2C4, petuzumab), TAK-165, GW-572016 (lonafarnib), GW-282974, EKB-569, PI-166, dHER2 (HER2 Vaccine), APC8024 (HER2 Vaccine), anti-HER/2neu bispecific antibody, B7.her2IgG3, AS HER2 trifunctional bispecfic antibodies, mAB AR-209 and mAB 2B-1.

Examples of alkylating agents include, but are not limited to, nitrogen mustard N-oxide, cyclophosphamide, ifosfamide, trofosfamide, Chlorambucil, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, KW-2170, mafosfamide, and mitolactol, carmustine (BCNU), lomustine (CCNU), Busulfan, Treosulfan, Decarbazine, Temozolomide, mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin.

Examples of antimetabolites include but are not limited to, methotrexate, 6-mercaptopurine riboside, mercaptopurine, 6-thioguanine, uracil analogues such as 5-fluorouracil (5-FU) alone or in combination with leucovorin, 5-fluorouracil decarbazine, tegafur, UFT, doxifluridine, carmofur, cytarabine, cytarabine, enocitabine, S-I, Alimta (premetrexed disodium, LY231514, MTA), Gemzar (gemcitabine), fludarabine, 5-azacitidine, capecitabine, cladribine, clofarabine, decitabine, eflornithine, ethnylcytidine, cytosine arabinoside, hydroxyurea, TS-I, melphalan, nelarabine, nolatrexed, ocfosate, disodium premetrexed, pentostatin, pelitrexol, raltitrexed, triapine, trimetrexate, vidarabine, vincristine, vinorelbine, mycophenolic acid, tiazofurin, Ribavirin, EICAR, hydroxyurea and deferoxamine.

Examples of antibiotics include intercalating antibiotics but are not limited to, aclarubicin, actinomycins such as actinomycin D, amrubicin, annamycin, adriamycin, bleomycin a, bleomycin b, daunorubicin, doxorubicin, elsamitrucin, epirbucin, glarbuicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, one or more agents selected from the group consisting of aclarubicin, amonafide, belotecan, cam ptothecin, 10-hydroxycamptothecin, 9-aminocam ptothecin, diflomotecan, irinotecan HCL (Camptosar), edotecarin, epirubicin (Ellence), etoposide, exatecan, gimatecan, lurtotecan, orathecin (Supergen), BN-80915, mitoxantrone, pirarbucin, pixantrone, rubitecan, sobuzoxane, SN-38, tafluposide and topotecan.

Examples of antibodies include, but are not limited to, Rituximab, Cetuximab, Bevacizumab, Trastuzimab, specific CD40 antibodies and specific IGFIR antibodies,

Examples of hormonal therapies include, but are not limited to, exemestane (Aromasin), leuprolide acetate, anastrozole (Arimidex), fosrelin (Zoladex), goserelin, doxercalciferol, fadrozole, formestane, tamoxifen citrate (tamoxifen), Casodex, Abarelix, Trelstar, finasteride, fulvestrant, toremifene, raloxifene, lasofoxifene, letrozole, flutamide, bicalutamide, megesterol, mifepristone, nilutamide, dexamethasone, predisone and other glucocorticoids.

Examples of retinoids/deltoids include, but are not limited to, seocalcitol (EB 1089, CB 1093), lexacalcitrol (KH 1060), fenretinide, Aliretinoin, Bexarotene and LGD-1550.

Examples of plant alkaloids include, but are not limited to, vincristine, vinblastine, vindesine and vinorelbine.

Examples of proteasome inhibitors include, but are not limited to, bortezomib (Velcade), MGI 32, NPI-0052 and PR-171.

Examples of immunologicals include, but are not limited to, interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-nl and combinations thereof. Other agents include filgrastim, lentinan, sizofilan, TheraCys, ubenimex, WF-10, aldesleukin, alemtuzumab, BAM-002, decarbazine, daclizumab, denileukin, gemtuzumab ozogamicin, ibritumomab, imiquimod, lenograstim, lentinan, melanoma vaccine (Corixa), molgramostim, OncoVAC-CL, sargaramostim, tasonermin, tecleukin, thymalasin, tositumomab, Virulizin, Z-100, epratuzumab, mitumomab, oregovomab, pemtumomab (Y-muHMFGI), Provenge (Dendreon), CTLA4 (cytotoxic lymphocyte antigen 4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include krestin, lentinan, sizofrran, picibanil and ubenimex.

Examples of pyrimidine analogs include, but are not limited to, 5-Fluorouracil,

Floxuridine, Doxifluridine, Ratitrexed, cytarabine (ara C), Cytosine arabinoside, Fludarabine, and Gemcitabine.

Examples of purine analogs include but are not limited to, Mercaptopurine and thioguanine.

Examples of antimitotic agents include, but are not limited to, paclitaxel, docetaxel, epothilone D (KOS-862) and ZK-EPO.

Examples of cytotoxic agents include, but are not limited to, such taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof;

Examples of targeted therapies that may be used include, but they are not limited to: hormone therapies (such as degarelix an luteinizing hormone-releasing hormone (LHRH) antagonist that reduces testosterone levels in prostate cancer), signal transduction inhibitors (such as imatinib and trastuzumab), as well as gene expression modulators (for example the HDAC inhibitors panobinostat and belinostat), apoptosis inducers (such as recombinant human TNF-related apoptosis-inducing ligand (TRAIL)) and angiogenesis inhibitors (such as sorafenib, sunitinib, pazopanib and everolimus).

Examples of immunotherapy agents that may be used include: monoclonal antibodies treatment (anti-CTLA4, anti-PD1), and chimeric antigen receptors (CARs)-T-Cells.

In embodiments there is disclosed a kit for performing the methods of the present invention which comprises

a) a sensitized cell,

b) instructions on how to perform the method.

According to another embodiment, the kit may also contain control biological fluids and and/or reagents to be used as negative and positive controls in the methods of the present invention.

These biological fluids and/or reagents may be, for example, useful for determining the predetermined reference value.

According to another embodiment, the sensitized cell may be chosen from an immortalized cell, a normal cell with a single oncosuppressor gene mutation, a normal cell with a single oncosuppressor gene decreased gene expression, a normal cell with a single activating mutation in a protooncogene, a normal cell with a single protooncogene increased gene expression. Preferably, the sensitized cell is a HEK 293 cell.

The present invention will be more readily understood by referring to the following examples which are given to illustrate the invention rather than to limit its scope.

### Example 1

### Blood Samples Collection and Serum Preparation

Cancer patient blood samples are accessed via the Biobank of the Cancer Research Program at the Glen Hospital, Montreal, Canada. Patients and healthy volunteers are recruited in the Department of General Surgery at the Royal Victoria Hospital, Glen Hospital, St. Mary's Hospital, Montreal, Canada, according to a protocol approved by the Ethics Committee of the institution. Blood samples are obtained with written consent from all participants. Serum is prepared, aliquoted and stored at −80° C. until use. Blood donor patients are categorized as followed:

Group 1. Blood collected from non-metastatic patients prior to primary tumor resection and sometime after surgery. If patients undergo chemotherapy blood is also drawn after chemotherapy ends.

Group 2. Blood collected from patients who have been cancer free for at least 2 years.

Group 3. Blood collected from patients at risk of developing cancer (due to familial history, or environmental exposure) and from patients undergoing tests to rule out neoplasia.

### Example 2

### Cell Culture, Serum Treatment and Analyses

HEK293 cells (ATCC) or BRCA mutated fibroblasts or any human cell line with single oncosuppressor mutation or protooncogene mutation are used as target “sensitized cells” to study their growth and malignant transformation. Cells are maintained according to the supplier's recommendations until 30% confluence at which point the different conditions are applied. All cultures are maintained for 2 weeks before analyses. At the end of the treatment period, cell transformation is studied by in vitro soft agar colony formation assay and in vivo tumor growth in NOD-SCID mice. Briefly:

Soft Agar Colony Formation (Anchorage Independent Cell Growth) Assay

Anchorage-independent cell growth is determined by analyzing the formation of colonies in soft agar. This in vitro assay is a hallmark of transformed cells. It determines the (i) incidence of colony formation, that is the frequency of cells able to grow and form colonies, and (ii) size distribution of these colonies, that reflects growth speed of cells in a given colony (i.e. the faster the cells grow, the bigger the colony they form). For this purpose, the size of all colonies in a given culture condition is determined using ImageJ™ Software. The values obtained are then categorized to compare one culture condition to another.

Soft agar assays are conducted in 12-well plates in semi-solid media. After trypsinization, 5000 cells are suspended in 10% FBS-supplemented DMEM medium containing 0.3% noble agar. This suspension is layered on top 0.8% agar-containing medium. Colonies (containing at least 50 cells) are scored and photographed after 3-4 weeks of culture under an inverted microscope (Evos XL AMG, Fisher Scientific™)

In Vivo Tumor Growth

Five-week-old female NOD-SCID mice (Jackson Laboratory) are used in compliance with McGill University Health Centre Animal Compliance Office (Protocol 2012-7280). Cells growing in log phase are harvested by trypsinization and washed twice with HBSS. Mice are injected subcutaneously with 2·10⁶ cells in 200 μl HBSS/Matrigel. When possible, mice are injected in both flanks to reduce the number of animals used in compliance with the “Three Rs” principles of the Animal Care Committee. Tumor growth is monitored regularly in all animals and once palpable masses are detected, the diameter is recorded with a caliper and volume estimated using the following formula V=a×b²×(π/6) (where a=major diameter; b=minor diameter and V=volume). Animals are euthanized by cervical dislocation when the tumor was cm in diameter. The resulting xenotransplants are photographed and processed as indicated below.

These parts of the study are performed at the Cancer Research Centre of the Glen site-McGill University Health Centre, Montréal, Canada.

### Example 3

### Tests of Blood Obtained from Patient Prior to Primary Cancer Resection and after Surgery

Blood is collected from patients, before undergoing primary cancer resection and after surgery.

After performing the HEK293 assay, Fibroblasts or any single oncosuppressor protooncogene mutated cell with both blood samples (prior and after surgery), the results is compared. Persistence of the malignant transformation of HEK293 cells after exposure to serum post-surgery indicates the persistence in the serum of putative oncogenic factors not cleared by the surgical resection. This finding is verified to see if it mirrors a probable lymphonodal involvement seen in the TNM staging (N1-2 stage) of the final pathology and follow the patient clinically to check if any recurrence occurs.

A statistical analysis is performed to verify the accuracy of the assay in predicting the nodal status of the patients, the sensitivity and specificity in the determining the rate of curative resections and the accuracy in anticipating recurrences.

### Example 4

### Tests of Blood Obtained from Patient Cancer Free for at Least Two Years

In this study, blood is collected from patients who have been cancer free for at least 2 years. Serum of all studied patients with metastases transform HEK293 cells into malignant cells. Furthermore, preliminary data suggest that the test might be positive also in patients at risk to develop metastases, since analysis of the sera of a few patients, studied with the HEK293 assay, anticipated metastatic disease 1 year prior to its diagnosis. This finding implies that the present method could be used as a reliable indicator of risk of metastatic recurrence. The scientific soundness of this assumption may be validated on a larger scale testing a higher number of cancer free patients at different time points, to correlate the results of the HEK 293 assay with the results of clinical tests, done during clinical follow up to rule out recurrence. To prove or negate the validity of this hypothesis, serum is collected from these cancer free patients and the HEK293 test is performed to monitor the response. In the case of positive test (transformation of the cells in vitro and in vivo) the patient will be identified and checked for any recurrence. Positive predictive value and negative predictive values will be calculated as well as sensitivity and specificity of the test.

### Example 5

### Detection of Early Stage Cancers

The serum of 2 patients with early stage cancers (T1 and T2) and negative tumor markers was able to transform the HEK 293 cells into malignant cells. This suggests that the method of the present invention might be also be utilized as a blood screening test for early diagnosis of cancer. To validate this assumption blood is collected from patients at risk for developing cancer and from patients undergoing tests such as such as serum tumor markers, mammogram, colonoscopy, total body imaging to rule out neoplasia.

The results obtained with the assay are compared to the results of the clinical tests, to verify whether they match. If they do match, estimation of the statistical sensitivity and specificity of the test will be performed with appropriate statistical tools and analysis.

### Example 6

### Case Information 1

For preparation of the sensitized cells, fibroblasts were infected with empty plasmid (PX458) or plasmid carrying a single guided (sg) BRCA1 construct. Cells were treated with control serum (Pooled Human Serum), Fetal bovine serum (FBS) or patient serum (case219), and the cells were transplanted in mice. The mice were euthanized 30 days later, and the tumor development were assessed (FIG. 4). Fibroblasts with PX458 treated with PHS or serum from Case219, and fibroblasts with sgBRCA1 treated with PHS displayed no tumors. Fibroblasts with sgBRCA1 treated with serum from Case219 displayed tumor.

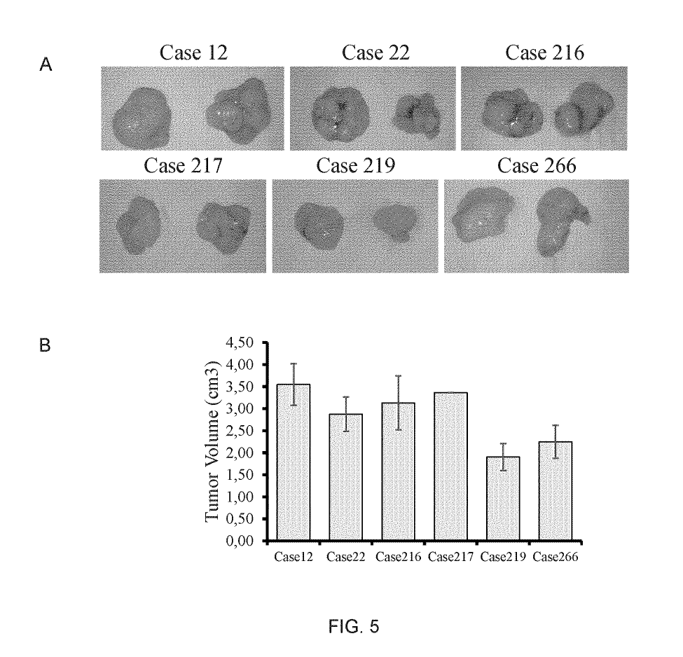

Next, blood samples were collected as described above in Example 1, and subjected to the assays described in Example 2, with the sensitized cells (i.e. target cells) being either sgBRCA1 fibroblasts. FIG. 5 shows in A) the tumors grown from in vivo tumor growth assays, and in B) the measured tumor volumes. Cases ID are matched to their respective cancer diagnosis as follows:

| Cases ID | Description | Target cells |
|---|---|---|
| Case 12 | Adrenal Carcinoma + lung metastasis | BRCA 1 |
| | | mutated |
| | | Fibroblasts |
| Case 22 | Breast cancer, lung + liver metastasis. | BRCA 1 |
| | | mutated |
| | | Fibroblasts |
| Case 216 | Metastatic neuroendocrine carcinoma | BRCA 1 |
| | | mutated |
| | | Fibroblasts |
| Case 217 | Breast cancer + liver metastasis | BRCA 1 |
| | | mutated |
| | | Fibroblasts |
| Case 219 | Colorectal cancer + liver metastasis | BRCA 1 |
| | | mutated |
| | | Fibroblasts |
| Case 266 | Anal Squamous Cell Carcinoma + liver | BRCA 1 |
| | metastasis | mutated |
| | | Fibroblasts |

Histopathology Analysis of BRCA 1 Mutated/Knocked Down Fibroblasts after Exposure to Serum of Cancer Patients. Case 219 (Colon Cancer)

| Case ID | CEA-P | CK7 | CK20 | CDX-2 | Ki67 | AE1/AE3 | Vimentin | CD34 |
|---|---|---|---|---|---|---|---|---|
| Case219 | ++ | Focal + | +++ | ++ | 85% | +++ | − | − |

Interestingly, the above results show that the sgBRCA1 fibroblasts differentiation toward intestinal adenocarcinoma appears more convincing than the effect on 293 cells (CEA-P, CK20, CDX-2, and AE1/AE3 positivity). It is noteworthy that vimentin, which is normally highly expressed in fibroblasts, is not expressed in the sgBRCA fibroblasts after exposure to cancer serum. This suggests that these cells are changing fate. 85% of cells are Ki67 positive and therefore appear to be proliferating.

### Example 8

### Case Information 2

Blood samples were collected as described above in Example 1, and subjected to the assays described in Example 2, with the sensitized cells (i.e. target cells) being HEK293 cells. FIG. 5 shows in A) the tumors grown from in vivo tumor growth assays, and in B) the measured tumor volumes. Cases ID are matched to their respective cancer diagnosis as follows:

| Cases ID | Description | Patient | Target cells |
|---|---|---|---|
| 300914 | Colon Cancer | SH | 293 cells |
| O71114 | Colon Cancer | BO | 293 cells |
| 101214 | Colon Cancer | MA | 293 cells |
| 150115 | Colon Cancer | VE | 293 cells |
| 200115 | Rectal Cancer | RA | 293 cells |
| 200115PM | Colorectal Cancer | VL | 293 cells |
| 140315 | Colon Cancer | SC | 293 cells |
| 160315 | Colorectal Cancer | CC | 293 cells |
| 160315PM | Colorectal Cancer | AR | 293 cells |
| 010415 | Colorectal Cancer | BA | 293 cells |
| 010415DH | Colon Cancer | VA | 293 cells |
| 150415 | Colon Cancer | FO | 293 cells |
| H220415 | Healthy Control | KS | 293 cells |
| 270415 | Colon Cancer | SH | 293 cells |
| 280415 | Colorectal Cancer-LM | CA | 293 cells |
| 040515 | Sigmoid Cancer | AM | 293 cells |
| 270515 | Colorectal Cancer | DO | 293 cells |
| 080615 | Muitorre Syndrome | FA | 293 cells |
| 090615 | Colon Cancer | BO | 293 cells |
| 010715 | Colonic Polyps | SO | 293 cells |
| 070715 | Colon Cancer | BI | 293 cells |
| 219 | Colorectal Cancer-LM | | 293 cells |

Histopathology Analysis

| Case ID | CEA-P | CK7 | CK20 | CDX-2 | Ki67 | AE1/AE3 | Vimentin | CD34 |
|---|---|---|---|---|---|---|---|---|
| 101214 | − | − | − | + | 100% | ++ | +++ | − |
| 150115 | − | − | − | Focal + | 100% | ++ | +++ | − |
| 200115 | − | − | − | Focal + | 100% | ++ | +++ | − |
| 200115PM | − | − | − | Focal + | 100% | ++ | +++ | − |
| 160315 | − | − | − | Focal + | 100% | ++ | +++ | − |
| 010415 | − | − | − | Focal + | 100% | ++ | +++ | − |
| 010415DH | − | − | − | Focal + | 100% | ++ | +++ | − |

The results above suggest that the HEK 293 cells differentiate to cancer but not to a specific type of cancer. They also suggest that the malignant transformation is toward carcinoma (AE1/AE3 positivity).

Only focal toward intestinal differentiation (CDX-2; only focal positivity)

Ki67 is 100% positive in all cells, suggesting that 100% of cells are proliferating.

### Example 9

### Exosome Staining

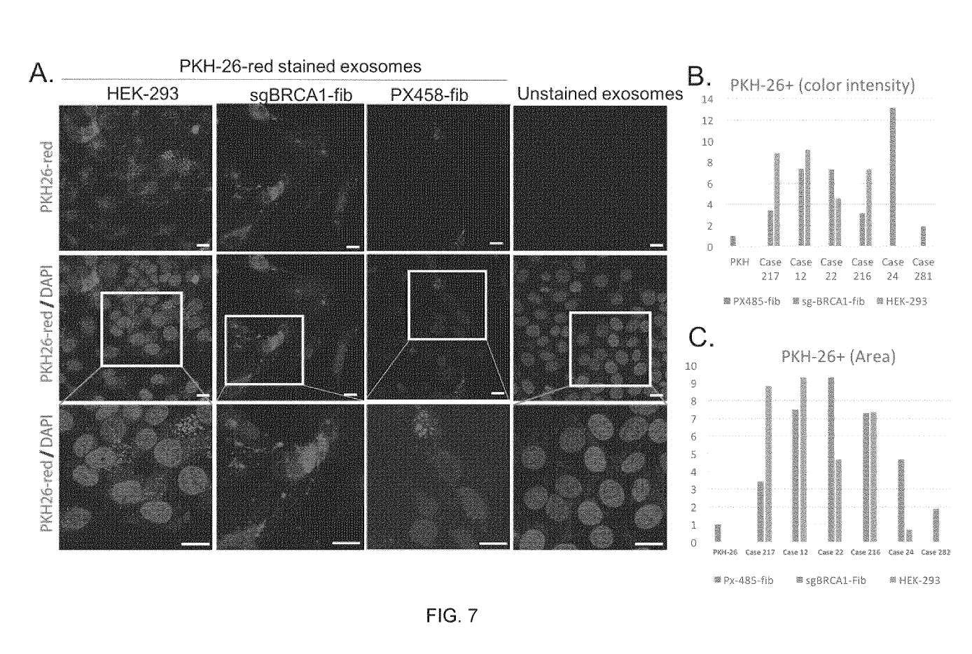

Now referring to FIG. 7 which illustrates the internalization of exosomes by sensitized cells. In panel (A) the staining with PKH-26 (red) shows the stained exosomes in HEK293 cells, fibroblasts infected with sgBRCA1 or empty PX458 vector, Also shown is nuclear DNA stained with DAPI. The staining intensity and area are quantified in panels (B) and (C) (using the ImageJ software) for PX458-fibroblasts, sgBRCA1 fibroblasts, and HEK293 cells treated or not with patient serum; The results show that the sensitized cells exposed to cancer serum internalized a greater number of cancer exosomes, suggesting, at least for the sgBRCA1 that oncosuppressor genes act by protecting cells from internalizing outside material that can induce genome instability.

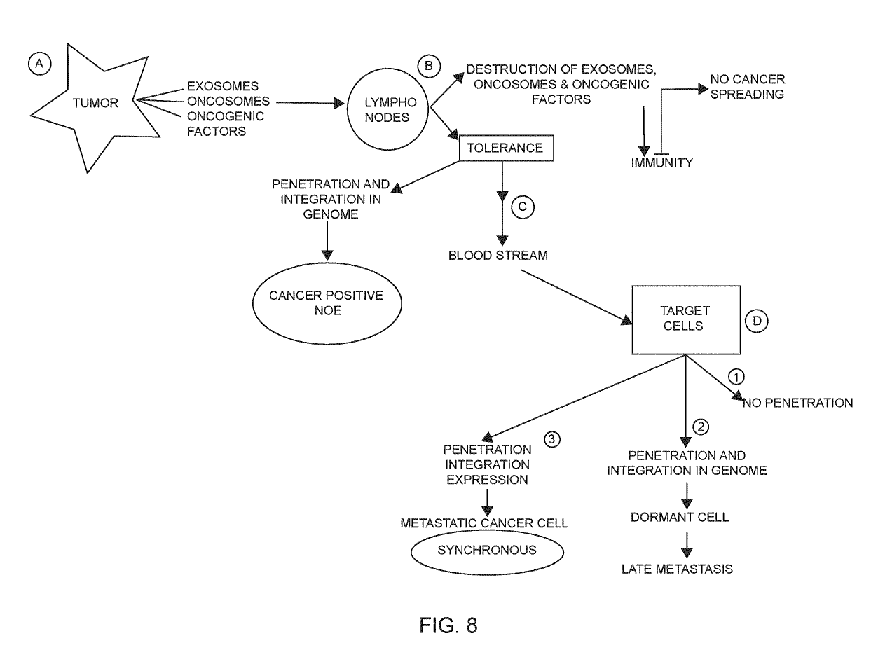

Now referring to FIG. 8, without wishing to be bound by theory, it is believe that the primary tumor produces oncogenic factors, exosomes, oncosomes, which all contain genetic material (DNA, RNA, mRNA, sRNA, iRNA, etc). These factors and substances enter the lymphatic system and arrive to the local lymph node. In the regional lymph nodes, these factors or substances either stimulate an immune response with development of lymphocytic clones that destroy the oncogenic material in the lymphnode or in the blood stream, or tolerance towards these factors is developed. If tolerance is developed these factors enter the cells in the lymph node and turn them into cancer cells (positive lymph node). Once tolerance is developed these factors travel into the blood stream and get anywhere in the body. Target cells in different organs are exposed to these substances. Different scenarios might occur:
a. The factors cannot penetrate the cells and therefore no metastases occur;
b. Penetration of these factors occurs and integration in the genome of the cells ensues. These factors once integrated do not get expressed but become part of the genome of the cells. The immune system controls and avoids the expression of these cancer genes or limits it. At a certain point in time due to weakening of the immune system or other factors these cancer genes get expressed and modify the genome of the cells turning the cells into cancer cells and give rise to what is called late metastases;
c. Penetration, integration, and expression of the cancer factors occur immediately and cells turns into cancer giving synchronous metastatic disease.

The assays of the present invention are able to identify these factors at any point during the process discussed above. Furthermore when the dormant cells become activated by intrinsic activation and expression of the cancer genes which were silenced, production of cancer factors occur with transformation of the cells into cancer and spread of the genes again, starting the cycle again and giving rise to metastases from the metastases.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCES

Abdouh M, Zhou S, Arena V, Arena M, Lazaris A, Onerheim R, Metrakos P, Arena G: Transfer of malignant trait to immortalized human cells following exposure to human cancer serum. J. Exp. Clin. Cancer Res. 2014, 33(1):86.
Abdel-Mageed Z Y, Yang Y, Thomas R, Ranjan M, Mondal D, Moraz K, Fang Z, Rezk B M, Moparty K, Sikka S C, Sartor O, Abdel-Mageed A B: Neoplastic reprogramming of patient-derived adipose stem cells by prostate cancer cell-associated exosomes. Stem Cells 2014, 32: 983-997.
Balaj L, Lessard R, Dai L, Cho Y J, Pomeroy S L, Breakefield X O, Skog J: Tumour microvesicles contain retrotransposon elements and amplified oncogene sequences. Nat Commun 2011, 2: 180.
Canadian Cancer Society. Cancer Statistics. 2015. http://www.cancer.ca/en/cancer-information/cancer-101/cancer-statistics-at-a-glance/?region=on
Canis M, Lechner A, Mack B, Zengel P, Laubender R P, Koehler U, Heissmeyer V, Gires O: CD133 induces tumour-initiating properties in HEK293 cells. Tumor Biol 2013, 34: 437-443.
Cohen S J, Punt C J, Iannotti N, Saidman B H, Sabbath K D, Gabrail N Y, Picus J, Morse M A, Mitchell E, Miller M C, Doyle G V, Tissing H, Terstappen L W, Meropol N J: Prognostic significance of circulating tumor cells inpatients with metastatic colorectal cancer. Ann Oncol. 2009, 20: 1223-1229.
Gaiffe E, Pretet J L, Launay S, Jacquin L, Saunier M, Hetzel G, Oudet P, Mougin C: Apoptotic HPV Positive Cancer Cells Exhibit Transforming Properties. PLOS ONE 2012, 7:e36766.
Garcia-Olmo D, Garcia-Olmo DC, Ontanon J, Martinez E, Vallejo M: Tumor DNA circulating in the plasma might play a role in metastasis. The hypothesis of the genometastasis. Histol Histopathol 1999, 14: 1159-1164
Garcia-Olmo DC, Domínguez C, Garcia-Arranz M, Anker P, Stroun M, García-Verdugo J M, Garcia-Olmo D: Cell-Free Nucleic Acids Circulating in the Plasma of Colorectal Cancer Patients Induce the Oncogenic Transformation of Susceptible Cultured Cells. Cancer Res 2010, 70:560-567.
Grant R, Ansa-Addo E, Stratton D, Antwi-Baffour S, Jorfi S, Kholia S, Krige L, Lange S, Inal J: A filtration-based protocol to isolate human plasma membrane-derived vesicles and exosomes from blood plasma. J Immunol Methods 2011, 371: 143-151.
Hamid T, Malik M T, Kakar S S: Ectopic expression of PTTG1/securing promotes tumorigenesis in human embryonic kidney cells. Mol Cancer 2005, 4: 1-13.
Hood J L, San R S, Wickline S A: Exosomes released by melanoma cells prepare sentinel lymph nodes for tumor metastasis. Cancer Res 2011, 71: 3792-3801.
Lalmahomed Z S, Mostert B, Onstenk W, Kraan J, Ayez N, Gratama J W, Grunhagen D, Verhoef C, Sleijfer S: Prognostic value of circulating tumour cells for early recurrence after resection of colorectal liver metastases. Br. J. Cancer. 2015. 112: 556-561.
Lin Y L, Han Z B, Xiong F Y, Tian L Y, Wu X J, Xue S W, Zhou Y R, Deng J X, Chen H X: Malignant transformation of 293 cells induced by ectopic expression of human Nanog. Mol Cell Biochem 2011, 351: 109-116.
Louis N, Evelegh C, Graham F L: Cloning and sequencing of the cellular viral junctions from the human adenovirus type 5 transformed 293 cell line. Virology 1997, 233: 423-429.
Peinado H, Alečković M, Lavotshkin S, Matei I, Costa-Silva B, Moreno-Bueno G, Hergueta-Redondo M, Williams C, Garcia-Santos G, Ghajar C, Nitadori-Hoshino A, Hoffman C, Badal K, Garcia B A, Callahan M K, Yuan J, Martins V R, Skog J, Kaplan R N, Brady M S, Wolchok J D, Chapman P B, Kang Y, Bromberg J, Lyden D: Melanoma exosomes educate bone marrow progenitor cells toward a prometastatic phenotype through MET. Nat Med 2012, 18: 883-891.
Rahbari N N, Aigner M, Thorlund K, Mollberg N, Motschall E, Jensen K, Diener M K, Buchler M W, Koch M, Weitz J: Meta-analysis shows that detection of circulating tumor cells indicates poor prognosis in patients with colorectal cancer. Gastroenterology. 2010, 138: 1714-1726.
Runz S, Keller S, Rupp C, Stoeck A, Issa Y, Koensgen D, Mustea A, Sehouli J, Kristiansen G, Altevogt P: Malignant ascites-derived exosomes of ovarian carcinoma patients contain CD24 and EpCAM. Gynecol Oncol 2007, 107: 563-571.
Tewes M, Kasimir-Bauer S, Welt A, Schuler M, Kimmig R, Aktas B: Detection of disseminated tumor cells in bone marrow and circulating tumor cells in blood of patients with early-stage male breast cancer. J Cancer Res. Clin. Oncol. 2015, 141 (1): 87-92.
Trejo-Becerrill C, Perez-Cardenas E, Taja-Chayeb L, Anker P, Herrera-Goepfert R, Medina-Velazquez L A, Hidalgo-Miranda A, Perez-Montiel D, Chavez-Blanco A, Cruz-Velazquez J, Diaz-Chavez J, Gaxiola M, as-Gonzalez AD: Cancer Progression Mediated by Horizontal Gene Transfer in an In Vivo Model. PLOS ONE 2012, 7 (12):e52754.
Venugopal C, Wang X S, Manoranjan B, McFarlane N, Nolte S, Li M, Murty N, Siu K W M, Singh S K: GBM secretome induces transient transformation of human neural precursor cells. J Neurooncol 2012, 109:457-466.

1. (canceled)

2. A method for treatment of cancer in a patient comprising:
a) determining with a biological assay an oncogenic potential of a sensitized cell contacted with a biological fluid derived from said patient, compared to a reference value; and
b) administering a treatment to said patient if the oncogenic potential of said sensitized cell is above or below a reference value,
wherein said sensitized cell is chosen from an immortalized cell, a normal cell with a single oncosuppressor gene mutation, a normal cell with a single oncosuppressor gene decreased gene expression.

3.-6. (canceled)

7. The method of claim 2, wherein said normal cell with a single oncosuppressor gene mutation is a BRCA mutated fibroblast.

8. The method of claim 2, wherein said normal cell with a single oncosuppressor gene decreased gene expression is a fibroblast with a decrease BRCA expression.

9. The method of claim 2, wherein said oncogenic potential of said sensitized cell is above said reference value, or wherein said oncogenic potential of said sensitized cell is below said reference value.

10. (canceled)

11. The method of claim 2, wherein said biological fluid derived from said patient is chosen from blood, serum, lymph, and a culture media contacted with a tumor from said patient.

12. The method of 2, wherein said biological assay is a soft agar colony formation/anchorage independent cell growth assay, an in vivo tumor growth assay, a cellular growth rate measurement assay, a cellular metabolic rate measurement assay, a cellular proliferation rate measurement assay, a biomarker expression measurement assay, a biomarker activity measurement assay, an exosome internalization assay, or a combination thereof.

13. The method of claim 12, wherein said soft agar colony formation/anchorage independent cell growth assay provides an increase of colony size of the sensitized cells contacted with said biological fluid derived from said patient, compared to a reference value from a control.

14. The method of claim 12, wherein said soft agar colony formation/anchorage independent cell growth assay provides an increase of the number of colonies of the sensitized cells contacted with said biological fluid derived from said patient, compared to a reference value from a control.

15. The method of claim 12, wherein said in vivo tumor growth assay provides an increased tumor diameter, an increased tumor volume, or both, at a given time, of the sensitized cells contacted with said biological fluid derived from said patient, compared to a reference value from a control at said given time.

16. The method of claim 12, wherein said cellular growth rate measurement assay provides an increased growth rate of said sensitized cell contacted with said biological fluid derived from said patient, compared to a reference value from a control.

17. The method of claim 12, wherein said cellular metabolic rate measurement assay provides an increased metabolic activity of said sensitized cell contacted with said biological fluid derived from said patient, compared to a reference value from a control.

18. The method of claim 12, wherein said cellular proliferation rate measurement assay provides an increased proliferation of said sensitized cell contacted with said biological fluid derived from said patient, compared to a reference value from a control.

19. The method of claim 12, wherein said biomarker expression measurement assay provides an increased expression of a biomarker or a decreased expression of a biomarker in said sensitized cell contacted with said biological fluid derived from said patient, compared to a reference value from a control.

20. The method of claim 12, wherein said biomarker activity measurement assay provides an increased activity of a biomarker or a decreased activity of a biomarker in said sensitized cell contacted with said biological fluid derived from said patient, compared to a reference value from a control.

21. The method of 2, wherein said treatment is chosen from a surgical intervention, administering a therapeutic agent, a radiotherapy treatment, and a combination thereof.

22. The method of claim 2, wherein said cancer is selected from the group consisting of breast cancer, colon cancer, pancreatic cancer, sarcoma, prostate cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, kidney cancer, bladder cancer, neuroendocrine tumor and liver cancer.

23. The method of claim 1, further comprising a step of cancer screening.

24. The method of claim 23, wherein said cancer screening is a serum tumor marker screening, a colonoscopy, a mammogram, a prostate exam, a PET scan, a CT scan, an MRI scan, an ultrasound scan, or combinations thereof.

25. The method of claim 24, wherein said patient is a patient having had a primary tumor resected.

26. A kit for performing the method of claim 2, comprising:
a) a sensitized cell,
b) instructions on how to perform the method.
